(19) United States
(12) Patent Application Publication
(10) Pub. No.: US 2002/0067801 A1
(43) Pub. Date: Jun. 6, 2002

(54) Flavored intraoral film holder

(76) Inventor: Valerie A. Gomez, Northglenn, CO (US)

Correspondence Address: Thomas W. Hanson, LLC, 3555 S. Sherman St., Suite 1, Englewood, CO, 80110, US

(21) Appl. No.: 09/730,105

(22) Filed: Dec. 5, 2000

(51) Int. Cl. G03C 5/16; A61B 6/14; G03B 42/02

(52) U.S. Cl. 378/169

(57) ABSTRACT

An improved holder, such as a bitewing loop, bitewing tab, or bite block, for intraoral x-ray film as is typically used in the dental field. The holder incorporates a flavoring substance on one or more of the exterior surfaces of the holder which are likely to come into contact with the patient's mouth. If desired, more than one flavor may be used on the same holder to provide variety. Alternatively, the film packet may also be flavored, in the same, or different, flavor as the holder.

### FIELD OF THE INVENTION

This invention relates to the field of holders, such as bitewings, for dental x-ray film.

### BACKGROUND OF THE INVENTION

Periodic visits to the dentist are often something which are endured rather than enjoyed since some of the procedures may be unpleasant. One such procedure is the taking of x-rays. This requires that the x-ray film be placed within the patient's mouth, behind the teeth to be imaged. A typical approach is to use a bitewing, or bite block, holder attached to the film packet on which the patient bites down to hold the film packet in place during the exposure.

The structure and placement of the packet and bitewing necessitates that they come in contact with various areas of the patient's mouth. This may include the teeth, gums, cheeks, and tongue. While the duration of this contact is brief, it is sufficient to transfer the taste of the holder and/or packet to the patient, who often complains that the taste is unpleasant.

In order to improve the overall experience for the patient, it would be preferable to mask or eliminate this unpleasant taste. One approach would be to flavor the holder or packet. Similar approaches have been taken with the polishing paste and floss used during the procedures and with the rubber gloves' worn by the dental staff.

There is a need for flavored holders and packets for intraoral x-ray film. Preferably this flavoring would be applied to all surfaces which come in contact with any part of the patient's mouth. To enhance the effect, the holder or packet could be color coded with a color which further suggests the flavor to the patient, strengthening the response.

### SUMMARY OF THE INVENTION

The present invention is directed to an improved holder for dental x-ray film in which a flavoring substance is applied to an exterior surface of the holder.

According to the invention there is provided a holder with a plurality of discrete exterior surfaces, with the flavoring applied to two or more of the surfaces.

According to an aspect of the invention more than one flavor may be applied to the surfaces of the holder.

According to another aspect of the invention the film packet may be flavored, either in combination with the holder or by itself.

The advantages of such an apparatus are an x-ray film carrier (holder and/or packet) which presents a pleasant flavor to the patient, rather than an unpleasant one, improving the patient's experience.

The above and other features and advantages of the present invention will become more clear from the detailed description of a specific illustrative embodiment thereof, presented below in conjunction with the accompanying drawings.

### BRIEF DESCRIPTION OF THE DRAWINGS

FIG. 1 illustrates a conventional bitewing loop holder and film packet held in position by the patient's teeth.

FIG. 2 provides an isometric view of an improved bitewing loop holder.

FIG. 3 provides a side view of an improved bitewing loop holder.

FIG. 4 provides an isometric view of an improved bitewing tab holder.

FIG. 5 provides a side view of an improved bitewing tab holder.

FIG. 6 provides an isometric view of an improved foam block holder.

FIG. 7 provides a side view of an improved foam block holder.

FIG. 8 provides an isometric view of a first version of an improved bite block holder with a film packet in place.

FIG. 9 provides a side view of a first version of an improved bite block holder without the film packet.

FIG. 10 provides an isometric view of a second version of an improved bite block holder with a film packet in place.

FIG. 11 provides a side view of a second version of an improved bite block holder without the film packer.

### DETAILED DESCRIPTION OF THE INVENTION

The following discussion focuses on the preferred embodiment of the invention, in which flavoring is added to holders for intraoral x-ray film. However, as will be recognized by those skilled in the art, the disclosed method and apparatus are applicable to a variety of similar situations.

Glossary

The following is a brief glossary of terms used herein. The supplied definitions are applicable throughout this specification and the claims unless the term is clearly used in another manner.

Holder—generally any device intended to retain x-ray film in position within the patient's mouth. While illustrated in use with holders on which the patient bites, use with other types of holders is anticipated.

Packet—generally the sleeve which holds the x-ray film itself. The packet is commonly constructed of vinyl or paper.

Preferred Embodiment

The disclosed invention is described below with reference to the accompanying figures in which like reference numbers designate like parts. Generally, numbers in the 200's refer to prior art elements or elements in the surrounding environment while numbers in the 100's refer to elements of the invention.

Overview

Film holders are available in a variety of configurations to serve the needs of the dental community. They differ primarily in how they are attached to the film packet and the position in which they hold the film relative to the patient's teeth. The more common configurations are discussed briefly below.

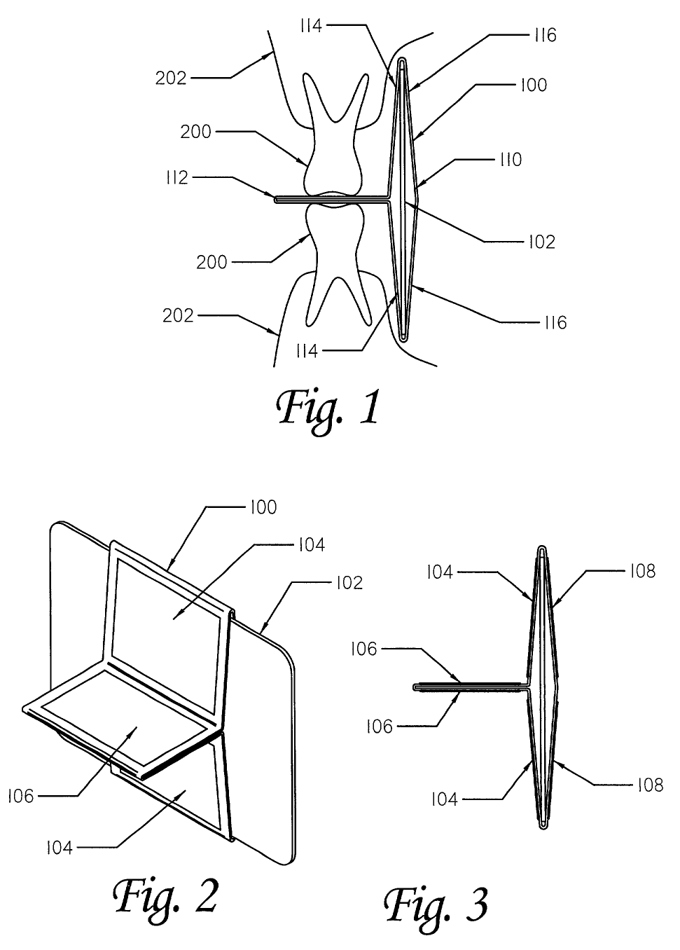

FIGS. 1-3 illustrate a bitewing loop holder, 100. The holder forms a closed loop, 110, into which the film packet, 102, is inserted. A lateral projection, 112, is intended to be clamped between the patient's teeth, 200, to hold the holder and film in position. The outer surfaces, 114, of the holder will typically contact the patient's gums, 202, while the inner surfaces, 116, likely contact the tongue.

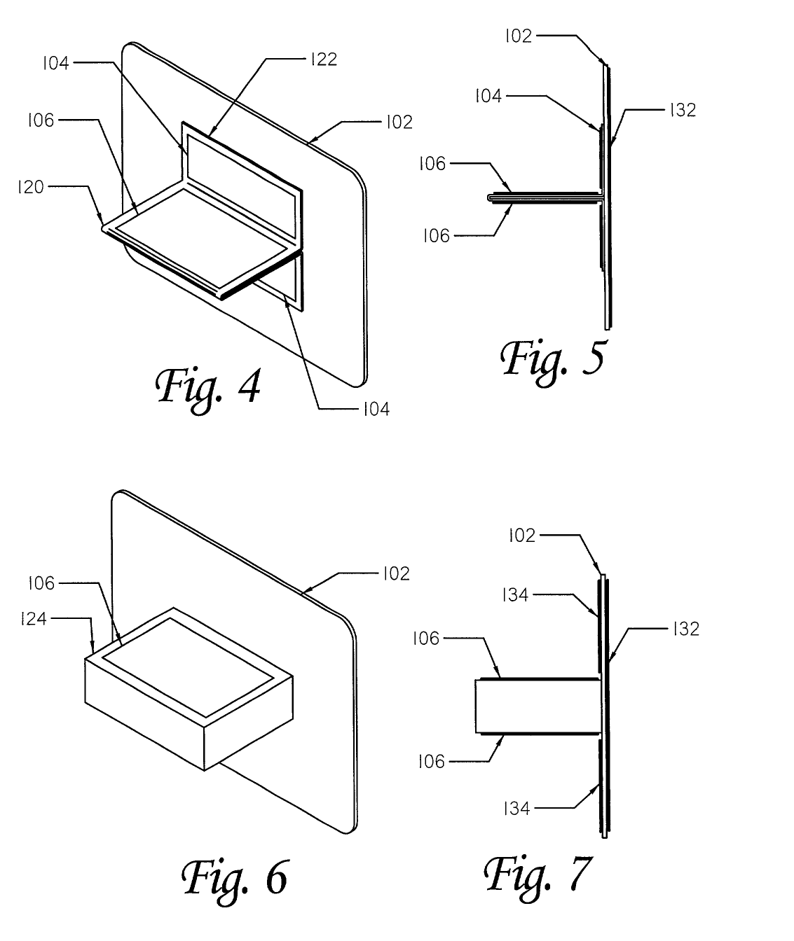

A similar bitewing tab configuration, 120, is illustrated in FIGS. 4 & 5. This configuration differs in that there is no loop for the film packet. Instead, the bitewing tabs, 122, are adhesively bonded to the face of the film packet, 102. Here, the holder may contact the teeth and gums, while the packet may contact the tongue.

The bitewing holder, 124, of FIGS. 6 & 7 is also adhesively bonded to the packet, 102. Rather than a folded structure, this holder is a relatively dense foam block. The holder will likely only contact the patient's teeth, and possibly the inside of cheek, while the packet will contact the gums and tongue.

The bitewing tab and foam block forms of holder are typically supplied separately from the film packet and are affixed immediately prior to use.

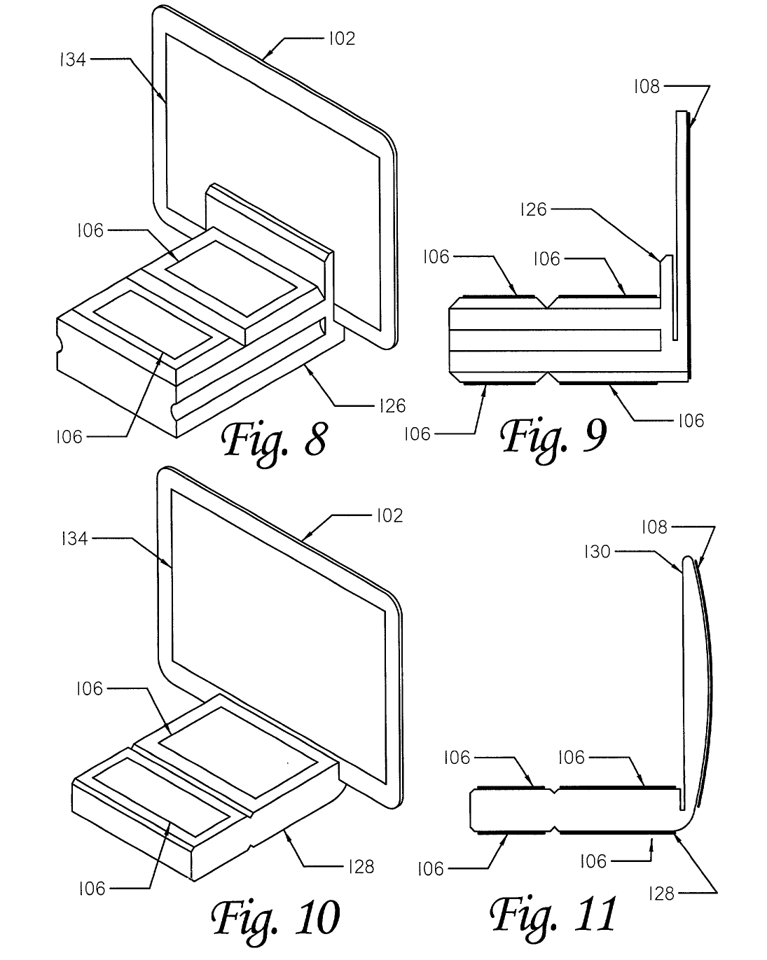

FIGS. 8 & 9 illustrate a first bite block configuration, 126, and FIGS. 10 & 11 illustrate a second bite block configuration, 128. Each of these is used for anterior or posterior exposures where the film needs to be held adjacent a single row of teeth. Because the film extends away from the holder for most of its width, a supporting arm, 130, is provided for additional support. This arm may intrude significantly into the patients mouth and will almost certainly contact the gums, tongue, or both. Note that both configurations provide a transverse groove which allow the block to be snapped off to shorten it. Where this is not done, it may protrude well past the teeth and contact the patient's cheek or lips. Note that the embodiment of FIGS. 8 & 9 incorporates grooves along the side for the attachment of a position indicating device or aiming ring for use with a bisection of the angle technique or similar. While generally described herein as used for a bitewing application, the inventive holder should be understood to be applicable to all intraoral x-ray applications regardless of the specific technique used.

All of these film holder configurations share the characteristic that some or all of the surfaces may come into contact with the patient's mouth. The flavor of the holder may be sensed directly or indirectly, as when the patient runs their tongue over their teeth which were clamped on the bitewing.

Structure

The present invention comprises an improved holder, film packet, or combination thereof in which a flavored substance is applied to one or more surfaces which are likely to come into contact with the patient's mouth. See FIGS. 2 & 3. Flavoring, 106, applied to that portion of the holder on which the patient bites will transfer that flavor to the teeth from where it will be transferred to the tongue. Similarly, flavoring, 104, applied to the outer surfaces of the bitewing loop, 110, or tabs, 122, will transfer to the gums. Flavoring, 108, applied to the inner surface of the bitewing loop will transfer directly to the patient's tongue. Any or all of these exterior surfaces can be flavored.

The flavoring can be any of the various flavoring substances known in the art which are suitable for impregnating or coating the material used in the holder.

The flavoring can be applied to the holder using any of several well known techniques. The material itself may be impregnated with the flavor. This is especially suitable where it is desired to flavor all of the material and can be applied prior to forming or cutting the individual holders. The flavor may also be brushed, sprayed, or rolled on. This would be preferred where it is desired to apply the flavoring only to selected surfaces. This may be done during the process of manufacturing the holders or after they have been made. This approach could even be applied in the dentist's office on an as needed basis. The flavor may also be applied as a separate layer, distinct from the base material of the holder. This is especially applicable where the holder is made of vinyl or a similar material which does not absorb significant amounts of liquid. The flavoring could be applied as a liquid which then dries on the surface of the holder, or as a gel which adheres to the surface.

When using those holder configurations which leave a significant portion of the film packet exposed, such as that illustrated in FIGS. 6 & 7, it may be desirable to apply the flavoring to the film packet, either in addition to or in place of flavoring the holder. Flavoring, 132, can be applied to the inner facing surface of the packet or, 134, to the outer facing surface, which will contact the gums. This approach is equally applicable to the remaining configurations.

Alternative Embodiments

An alternative embodiment of the present invention incorporates color coding which is matched to the flavoring used. For example: green for mint; pink for bubble gum; red for cherry; etc. This color coding can serve either or both of two purposes. First, the color coding aids in identification and selection of the holder with the desired flavor. Second, the color can provide a visual enforcement of the flavor, resulting in increased perceived flavor by the patient.

If desired, more than one flavor could be used on a holder. Especially where individual surfaces are treated separately by rolling, brushing, etc. These could be compatible flavors (such as vanilla and cherry); contrasting flavors (such as peppermint and cinnamon); or any other scheme. This would further entertain and intrigue the patient because the perceived flavor would vary depending on which surfaces of the holder came into contact with the patient's mouth.

While the preferred form of the invention has been disclosed above, alternative methods of practicing the invention are readily apparent to the skilled practitioner. The above description of the preferred embodiment is intended to be illustrative only and not to limit the scope of the invention.

I/We claim:

1. An improved holder for an x-ray film packet, comprising:
(a) a structure, adapted to retain the film packet, having an exterior surface; and
(b) a flavoring substance applied to said exterior surface.

2. The improved film holder of claim 1 wherein said structure comprises plural discrete exterior surfaces and further comprising a flavoring substance applied to at least two of said exterior surfaces.

3. The improved film holder of claim 2 wherein said flavoring applied to said at least two surfaces comprises at least two distinct flavors.

4. An improved carrier for x-ray film comprising:
(a) a film packet adapted to receive the film, having an exterior surface;
(b) a holder adapted to retain said film packet, having an exterior surface; and
(c) a flavoring substance applied to at least one of said film packet and holder exterior surfaces.

5. The improved carrier of claim 4 further comprising a flavoring substance applied to both said packet exterior surface and said holder exterior surface.

6. The improved carrier of claim 4 wherein said holder comprises plural discrete exterior surfaces and further comprising a flavoring substance applied to at least two of said exterior surfaces.

7. The improved film holder of claim 6 wherein said flavoring applied to said at least two holder surfaces comprises at least two distinct flavors.

8. An improved packet for intraoral x-ray film, comprising:
(a) a sleeve like structure adapted to receive the film, having an exterior surface; and
(b) a flavoring substance applied to said exterior surface.
